(19) United States
(12) Patent Application Publication
(10) Pub. No.: US 2012/0225792 A1
(43) Pub. Date: Sep. 6, 2012

(54) Gene Expression Biomarkers in PAP Test Material for Assessing HPV Presence and Persistence

(75) Inventors: Kim E. Creek, Columbia, SC (US); Lucia A. Pirisi-Creek, Columbia, SC (US); Diego Altomare, West Columbia, SC (US); Fang Wan, Middleton, WI (US)

(73) Assignee: University of South Carolina, Columbia, SC (US)

(21) Appl. No.: 13/402,411

(22) Filed: Feb. 22, 2012

Related U.S. Application Data
(60) Provisional application No. 61/445,172, filed on Feb. 22, 2011.

(51) Int. Cl. C40B 30/04; C12Q 1/70

(52) U.S. Cl. 506/9; 435/5

(57) ABSTRACT

In accordance with certain embodiments of the present disclosure, a method for the diagnosis of persistent HR-HPV in an individual is provided. An expression level of at least one biomarker in a biological sample is determined. The expression level of the at least one biomarker is compared to an expression level of corresponding biomarker(s) in a comparative sample, wherein the comparative sample contains the at least one biomarker in a level indicative of persistent HR-HPV. Persistent HR-HPV infection is predicted in the individual based upon the comparison of the expression level of the at least one biomarker between the comparative sample and the biological sample.

### FEDERALLY SPONSORED RESEARCH

This invention was made with government support under National Institutes of Health Grant IP20MD001770-01 awarded by the National Center on Minority Health and Health Disparities and the DNA Microarray Facility at University of South Carolina was made with government support in part under National Institutes of Health Grant 5P20RR016461 awarded by the National Center for Research Resources. The government has certain rights in the invention.

### BACKGROUND

Cervical cancer incidence has steadily decreased in the U.S. over the past fifty years due to broadly available screening and follow-up programs that detect and treat abnormal cervical lesions, respectively, but still remains a considerable burden worldwide with nearly 300,000 deaths each year. A significant proportion of abnormal cervical lesions would spontaneously regress within 1 year if not treated—˜50% of low grade and 35% of high grade lesions, 43% of cervical intraepithelial neoplasia 2 (CIN2) and 32% of CIN3. These cervical abnormalities are routinely treated or studied by biopsy. Consequently, the current screening and follow-up protocols result in expensive, unnecessary and painful interventions, some of which have detrimental consequences for future pregnancies. The main and necessary etiologic agent in the development of cervical cancer is the infection of cervical cells with sexually-transmitted, high-risk human papillomaviruses (HR-HPVs), with HPV16 being the most prevalent type. Although most women will come in contact and get infected with HPV at some point during their lifetime, cervical cancer is not a frequent outcome of HR-HPV infection. Only women with persistent HPV infection are truly at risk of developing cervical cancer.

As such, a need exists for biomarkers that could predict persistent HR-HPV infections, the ones at higher risk of progressing into cervical cancer, by performing microarray gene expression profiling on RNA samples isolated from exfoliated cervical cells. These biomarkers, or molecular signature, would constitute a cheaper, non-invasive and more accurate tool for the triage of HR-HPV positive women for further treatment, reducing the drawbacks of the current screening and follow-up protocols.

### SUMMARY

In accordance with certain embodiments of the present disclosure, a method for the diagnosis of persistent HR-HPV in an individual is provided. The method includes obtaining a biological sample from an individual. An expression level of at least one biomarker in the biological sample is determined, wherein the at least one biomarker is chosen from GPAT2, SHF, IRF4, CD86, DDX24, FNBP1, HBEGF, IL6, A 32 P29814, A 24 P925882, A 24 P255415, MAGOHB, CFI, and CXCL14. The expression level of the at least one biomarker is compared to an expression level of corresponding biomarker(s) in a comparative sample, wherein the comparative sample contains the at least one biomarker in a level indicative of persistent HR-HPV. Persistent HR-HPV infection is predicted in the individual based upon the comparison of the expression level of the at least one biomarker between the comparative sample and the biological sample.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates certain graphs in accordance with the present disclosure;

FIG. 2 illustrates the result of cluster analysis in accordance with the present disclosure;

FIG. 3 illustrates certain graphs in accordance with the present disclosure; and

FIG. 4 illustrates the result of cluster analysis in accordance with the present disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

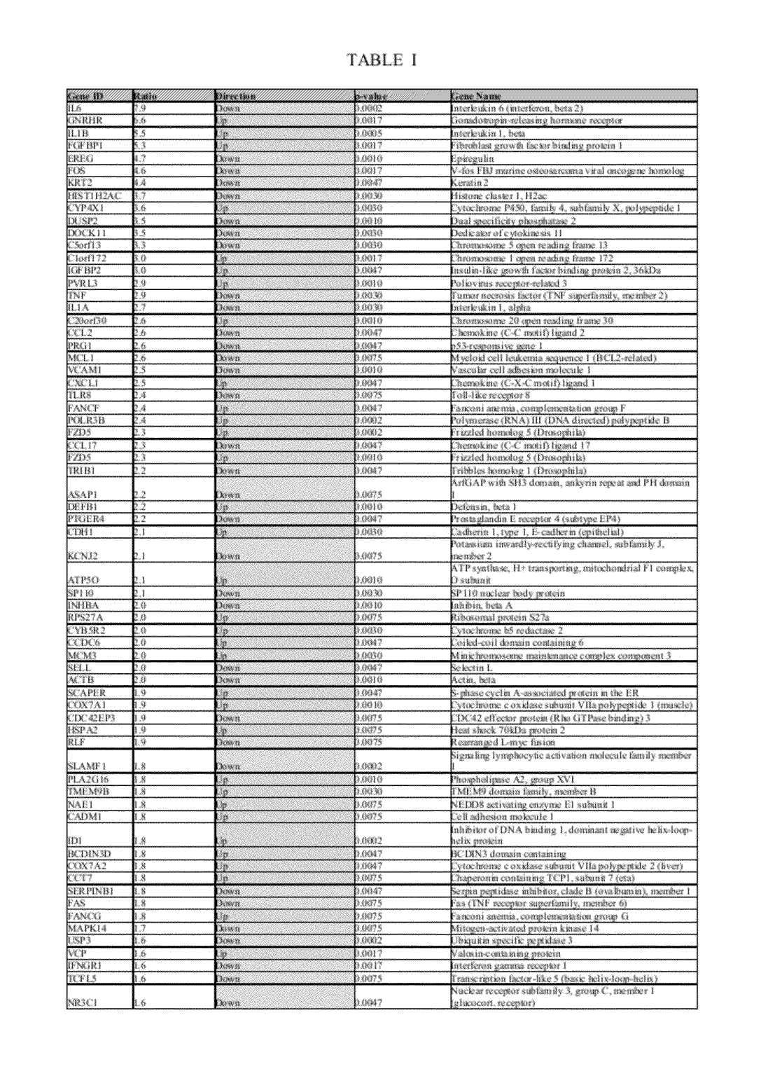

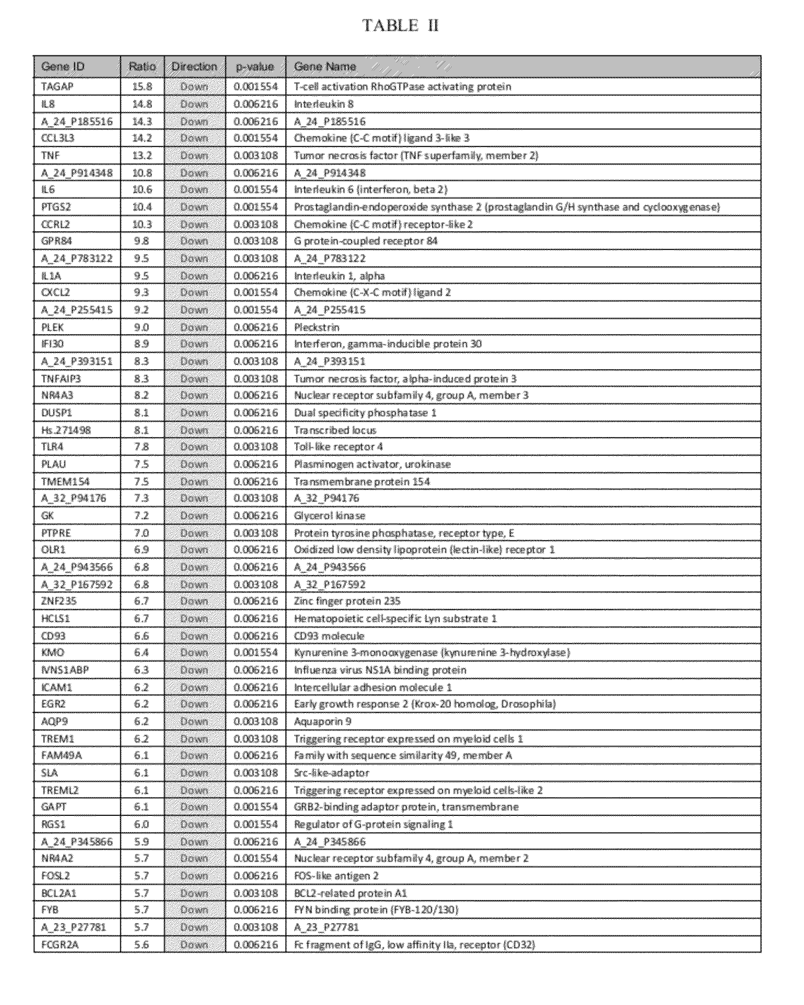

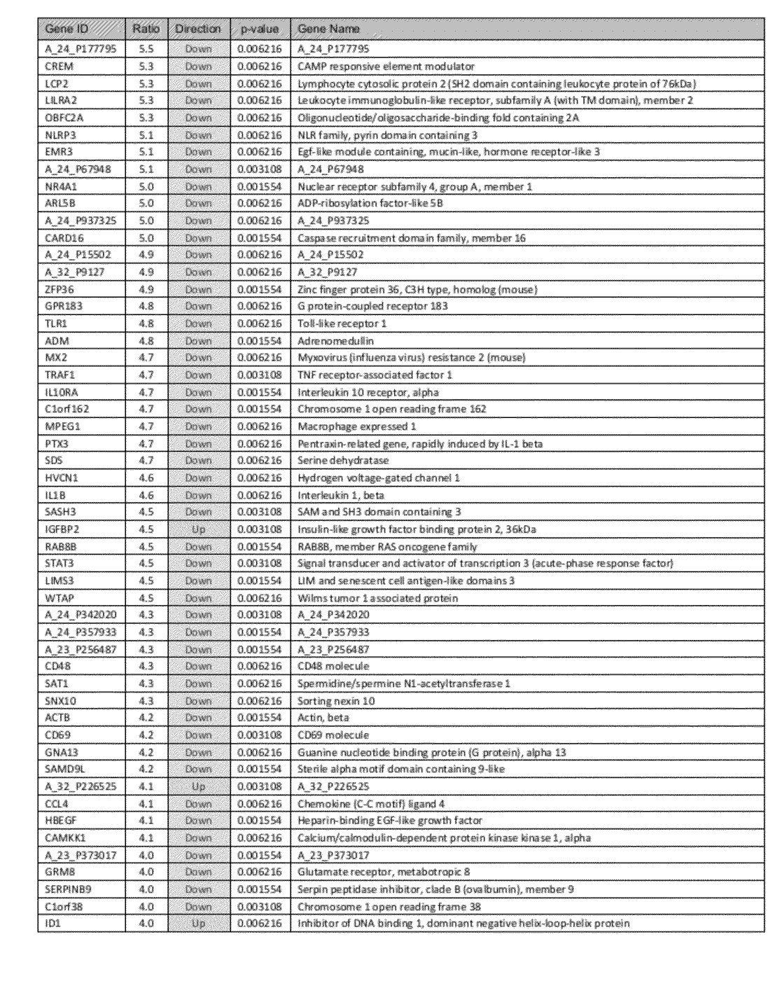

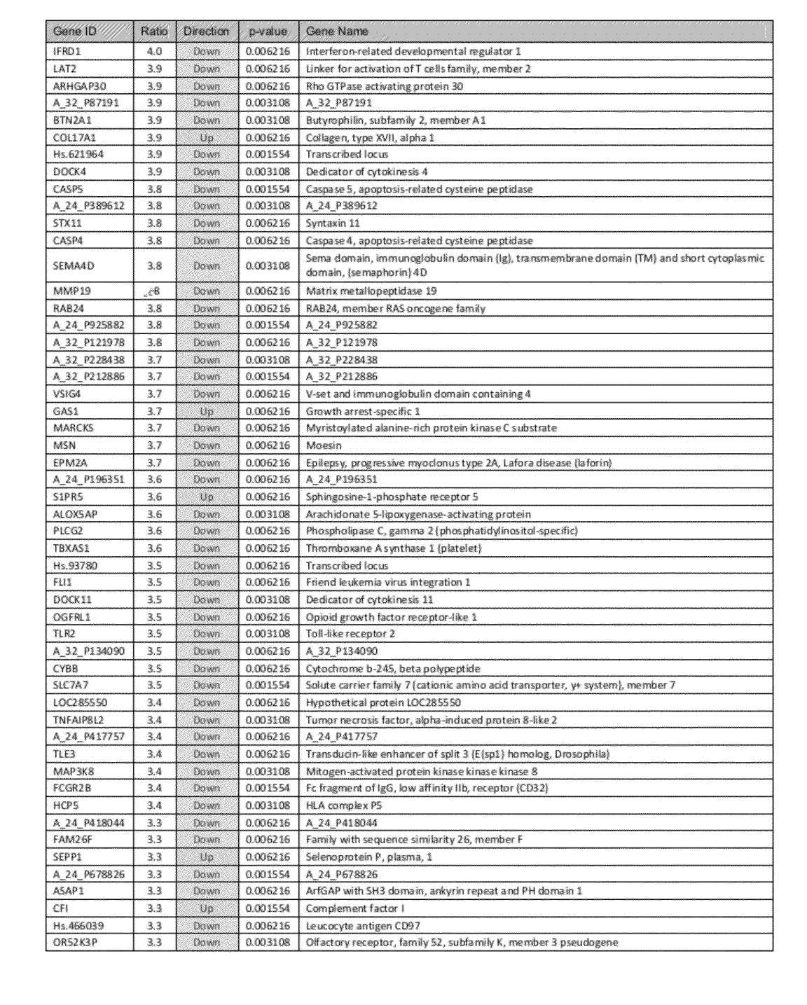

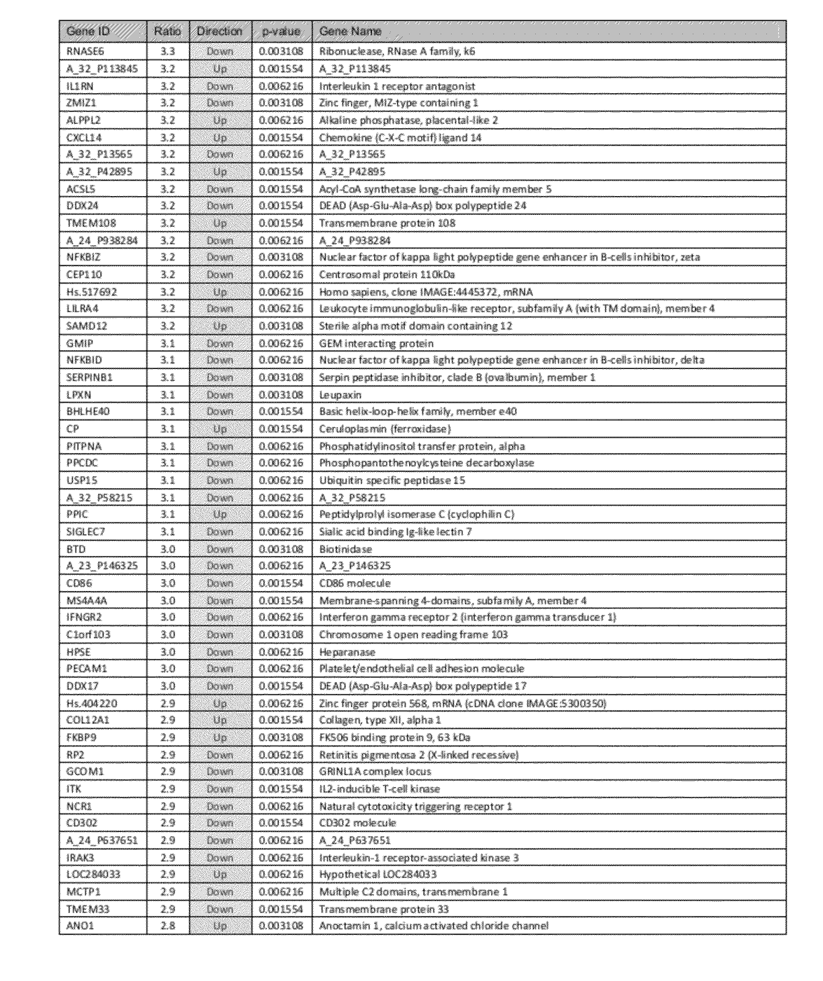

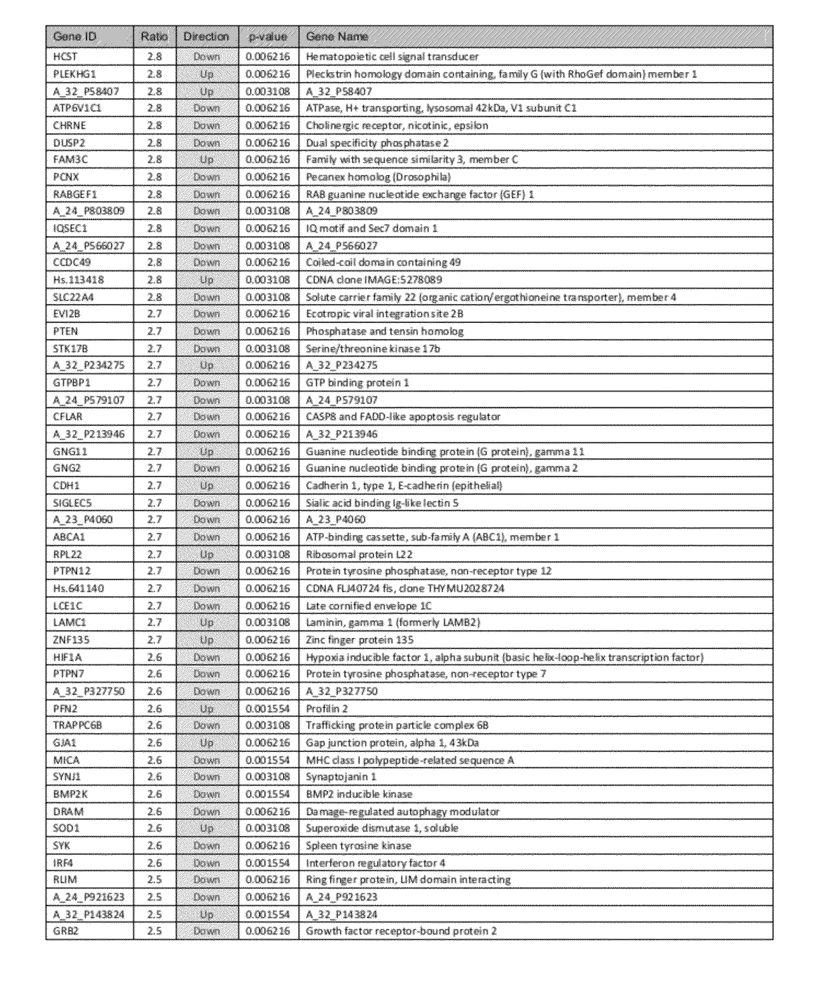

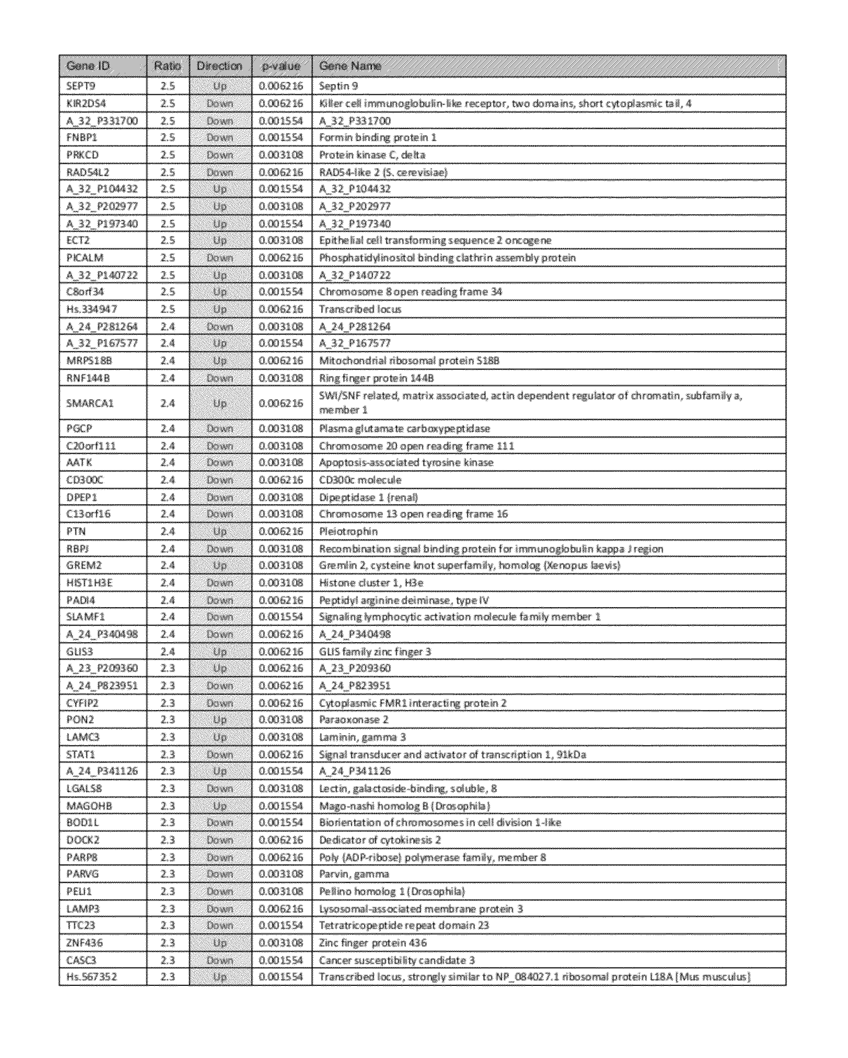

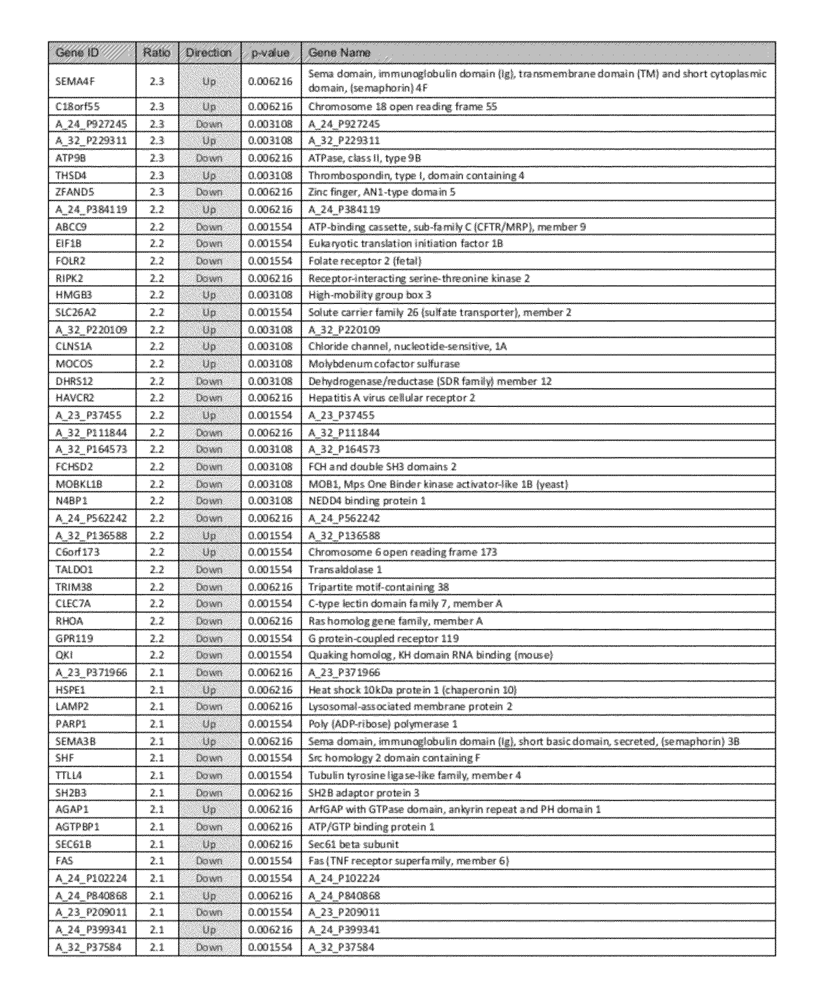

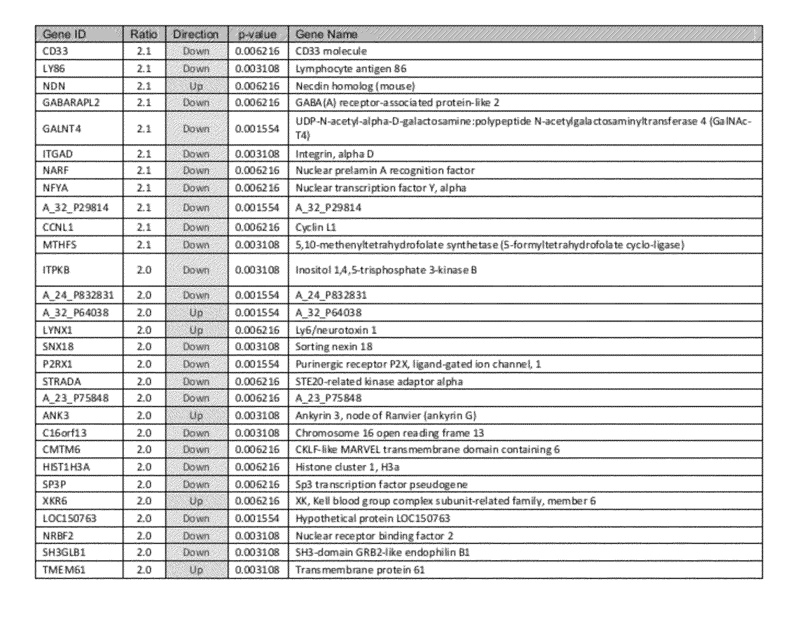

The term “biomarker” as used herein refers to a gene that is differentially expressed in individuals with persistent HR-HPV and is predictive of cervical cancer risk. The term “biomarker” includes one or more of the genes listed in FIGS. 2 and 4.

Accordingly, one aspect of the present disclosure is a method of diagnosing persistent HR-HPV in a subject by determining the expression of a biomarker in a biological sample from the subject, wherein the biomarker includes one or more biomarkers as shown in FIGS. 2 and 4, and comparing the expression of the biomarker with a control representative of persistent HR-HPV.

The term “diagnosing” as used herein refers to a method or process of determining whether a subject has persistent HR-HPV based on biomarker expression profiles.

The term “biological sample” as used herein refers to any fluid, cell or tissue sample from a subject which can be assayed for biomarker expression. In one embodiment, the test sample is a cell, cells or tissue from a biopsy from the subject. A preferred biological sample can include cells from a PAP test.

As used herein, the terms “control” and “standard” refer to a specific value that one can use to determine the significance of the value obtained from the sample. In one embodiment, a dataset may be obtained from samples from a group of subjects known to have persistent HR-HPV infection. The expression data of the biomarkers in the dataset can be used to create a control (standard) value that is used in testing samples from new subjects. In such an embodiment, the “control” or “standard” is a predetermined value for each biomarker or set of biomarkers obtained from subjects with persistent HR-HPV whose biomarker expression values are known.

The term “differentially expressed” or “differential expression” as used herein refers to a difference in the level of expression of the biomarkers that can be assayed by measuring the level of expression of the products of the biomarkers, such as the difference in level of messenger RNA transcript expressed or proteins expressed of the biomarkers. In a preferred embodiment, the difference is statistically significant. The term “difference in the level of expression” refers to an increase or decrease in the measurable expression level of a given biomarker as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 4, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed between a first sample and a second sample when the p-value is less than 0.1, preferably less than 0.05, more preferably less than 0.01, even more preferably less than 0.005, the most preferably less than 0.001.

In another embodiment, expression data from multiple biomarkers is analyzed using cluster techniques. In one embodiment, clustering is based on correlation of average normalized signal intensities. In one embodiment, the biomarkers comprise the biomarkers listed in FIGS. 2 and 4.

The phrase “determining the expression of biomarkers” as used herein refers to determining or quantifying RNA or proteins expressed by the biomarkers. The term “RNA” includes mRNA transcripts, and/or specific spliced variants of mRNA. The term “RNA product of the biomarker” as used herein refers to RNA transcripts transcribed from the biomarkers and/or specific spliced variants. In the case of “protein”, it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample, including microarrays, RT-PCR (including quantitative RT-PCR), nuclease protection assays and Northern blot analyses.

In addition, a person skilled in the art will appreciate that a number of methods can be used to determine the amount of a protein product of a biomarker of the invention, including immunoassays such as Western blots, ELISA, and immunoprecipitation followed by SDS-PAGE and immunocytochemistry.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

As described above, cancer of the cervix accounts for nearly 300,000 death per year and constitutes the second most common type of cancer among women worldwide. Epidemiologic and molecular studies have unequivocally established genital high-risk human papillomaviruses (HPV) as the primary etiologic agent in cervical cancer. HPV DNA has been found in cervical samples of virtually all (99.6%) patients with cervical cancer examined, with HPV type 16 (HPV16) being the most prevalent (54%). In addition, molecular studies of the high-risk HPV E6 and E7 oncoproteins have clearly demonstrated their involvement in cell transformation by targeting both p53 and RB tumor suppressor genes among other mechanisms.

Over 6 million persons are newly infected with genital HPV every year in the United States and most women (80%) will have contracted a sexually transmitted HPV infection by age 50. Nonetheless, cervical cancer is not a frequent outcome of HPV infection and only women with persistent viral infections are truly at higher risk of developing cervical cancer. While the incidence of cervical cancer in developed countries has been dramatically decreased since the advent of the Papanicolaou test, in undeveloped countries its occurrence is much higher. This has been possible due to the implementation of widely available screening program in wealthier countries where early precancerous lesions are detected and treated. However, current follow up and treatment protocols of both cytological and histological cervical abnormalities have important drawbacks that would be important to correct. The first of them is reflected by the fact that while LSIL and HSIL cytological results are followed with biopsies or mostly treated with excisional procedures, respectively, 40-50% of LSIL and 35% of HSIL will spontaneously regress and normalize cellular abnormalities. Similarly, despite regression rates of 43% for CIN2 and 32% for CIN3, all these histological lesions are treated by excisional or ablative procedures. Since there is not any other diagnostic tool useful to triage women at higher risk of developing malignancy, these interventions are necessary to prevent the evolution of some of the lesions into cancer of the cervix although they clearly constitute overtreatment of the women whose lesions would not have progressed if left untreated. Another important factor concerning the overtreatment issue is that ablative or excisional procedures have a negative impact on subsequent pregnancies doubling the risk of preterm delivery, a low-birth weight infant, or premature rupture of membranes. In addition, these treatment methods have morbidity and psychological impact factors associated with them, and are expensive. Furthermore, it has been reported that both cytological and histological diagnostic test have some reproducibility issues.

Given the limitations of the current diagnostic protocols for classification of women into follow-up and treatment groups, and since only women with persistent HR-HPV infections are actually at higher risk of developing cervical cancer, the identification of biomarkers that could predict which women will have persistent viral infection would dramatically improve the triage of patients into treatment or no-treatment groups therefore reducing the overtreatment that occurs at the present time.

In accordance with the present disclosure, methods of gene expression profiling are presented on RNA samples isolated from cervical exfoliated cells using microarray technology. Since no satisfactory methods to isolate good quality RNA from this type of cells had been reported, a successful optimization of the protocol was performed. Next, through analysis of samples from study participants that cleared HPV16 infections and from participants that had persistent HPV16 infections, a gene expression signature was identified that differentiates these conditions. In addition, by comparing gene expression profiles of samples from persistent and transient HPV infection to HPV negative samples it was observed that there is almost no gene expression difference between HPV16 persistent and HPV negative samples suggesting a lack of response to the HPV infection in HPV16 persisters. In contrast, the present disclosure describes that many differentially expressed genes when HPV negative samples were compared to HPV16 infected samples from women that later cleared the viral infection indicating that these patients do biologically respond to the virus. Finally, a comparison between cytologically normal and abnormal samples is described with the purpose of identifying biomarkers of these two conditions. Relevant biomarkers identified in accordance with the present disclosure include GNRHR, FZD5, FGFBP1, IL1B, POLR3B, PVRL3, EREG, IL6, USP3, GPAT2, SHF, IRF4, CD86, DDX24, FNBP1, HBEGF, A 32 P29814, A 24 P925882, A 24 P255415, MAGOHB, CFI, and CXCL14.

The present disclosure can be better understood with reference to the following examples.

### EXAMPLES

The primary goal of the examples undertaken herein was to investigate and identify the determinants of HPV persistence in women. Epidemiologic and molecular studies have unequivocally identified sexually-transmitted HR-HPVs as the main and necessary etiologic agent in the development of cervical cancer. Additional factors such as smoking, number of sexual partners, presence of other sexually transmitted infections, bearing a greater number of children and a suppressed immune system have also been proposed to increase the risk of developing cervical cancer. However, it is also well established that cervical cancer is not a frequent outcome of HR-HPV infection and only women with persistent viral infections are truly at higher risk of developing cervical cancer.

Most women will come in contact and get infected with HPV at some point during their lifetime, often at the onset of sexual activity, and some of these infections will result in an abnormal Pap tests. Whereas most women clear HPV infections without treatment, some may develop precancerous lesions that may later evolve into cervical cancer. In order to prevent the evolution of moderate and severe cervical abnormalities into cancer, current treatment guidelines in the medical community require further exploration through biopsies in the case of LSIL and through excisional treatment in the case of HSIL. In addition, moderated and severe cervical dysplasia (CIN2 and CIN3) found mostly in histological testing of biopsies of patients with LSIL cytology are also treated by ablational or excisional interventions. The procedures are painful, costly, and, in the case of excisional treatments, may lead to infertility later in life. Since a significant proportion of abnormal cervical lesions would spontaneously regress if not treated, the current protocols result in expensive, unnecessary and painful interventions. However, no accepted test exists to determine which patients require treatment and which do not. The main goal of the examples described herein is to identify novel biomarkers that can distinguish patients that require further follow up and treatment from the ones that do not.

HPV infection infrequently persists and progresses to cervical cancer and persistent infection with one of approximately 15 HR-HPVs is necessary for the development of cancerous precursors (CIN3) and cervical cancer. Therefore, prediction of the persistent infections using cervical exfoliated cells would constitute a cheaper, non-invasive and more accurate tool for the triage of women for further treatment. For this purpose, gene expression profiling using microarray technology was performed on RNA samples isolated from cervical exfoliated cells collected from HPV16 persistent Carolina Women's Care Study (CWCS) participants, the ones at higher risk of developing cervical cancer, and from CWCS participants with HPV16 infections that cleared. In order to be able to properly perform such experiments, optimization of the RNA isolation method has to be performed first since previous reported protocols yielded low quality RNA. In addition, the 2 previous sample groups were compared to HPV negative samples, which could give some insight regarding the gene expression changes due to HPV infection per se. Finally, cytologically normal and abnormal samples were also compared, with the aim of identifying biomarkers able to predict atypical cells in cervical exfoliated samples. The results of this work yielded a series of potential biomarkers for HPV persistence that, after proper validation, can find a use in the development of novel and more specific tests that will allow for the identification of women at the highest risk of HPV persistence and cervical neoplasia.

During pelvic examination cervical mucus and two cervical exfoliated cell (CEC) samples were collected. The first CEC sample was taken with a spatula and cytobrush, rinsed in 20 ml of PreservCyt solution (Cytyc, Cat. No. 0234004) and 15 ml was used in ThinPrep imaging for diagnostic cytology at LabCorp, and 5 ml was used for HPV testing/typing. The second CEC sample was also obtained with a spatula and cytobrush, the brush was rolled over the spatula in an effort to transfer the complete sample onto the brush and then placed into a 15 ml conical tube (Sarstedt, Cat. No. 62-554-002) containing 2 ml of the RNA stabilization solution RNAlater (Ambion, Part No. AM7021). The cytobrush's handle was cut off with scissors so that the cap will fit on the conical tube with the brush inside. This last sample was used for isolation of total RNA and gene expression profiling studies using microarrays. All samples were properly labeled and stored immediately after the exam in a −20° C. freezer until used. Additionally, a blood specimen was collected at the first visit. If a blood sample was not acquired on the first visit, the participant was asked to supply a saliva sample and the nurse practitioner made a second attempt on the following visit. Both blood clots and saliva samples were used for DNA isolation, which was used in single nucleotide polymorphism (SNP) analysis. Cervical mucus samples were used for cytokine profiling experiments.

Data generated from both surveys and the several laboratory tests were consolidated into an Access database. Through surveys, demographic information as well as the ethnicity of the participants was collected. In addition, information including stress, frequency and quantity of alcohol usage, tobacco and marijuana smoking, sexual behavior, exercise, diet, depression and medication or supplements being taken were also collected from the surveys. Among the tests that generated data uploaded into the database were a Papanicolaou (Pap) test, human papillomavirus (HPV) testing, HPV typing and cytokine quantitation. In addition, the database also contained information about unprocessed and processed samples including DNA and RNA specimens and cytokine extracts. The database proved critical for determining HPV persistence/clearance as well as for selecting and retrieving specific sets of samples for further study.

Cell suspensions in PreservCyt fluid (5 ml) contained in 15 ml conical tubes were centrifuged for 10 minutes at 3,000 rpm, the fluid was decanted and tubes were inverted and blotted on a paper towel. Cell pellets were resuspended in 1 ml of 100% ethanol and transferred to labeled 1.5 ml Eppendorf tubes. Subsequently, tubes were centrifuged for 10 minutes at 15,000 rpm, ethanol was removed by decantation and tubes were inverted and blot dried on paper towels. The tubes were placed in a DNA 110 SpeedVac (Savant, Cat No. BC-SDNA110) and dried for 5-10 minutes to remove all traces of the ethanol. The pellet was resuspended in 300 μl of proteinase K lysis buffer consisting of 10 mM Tris-HCl (pH 8.0), 5 mM EDTA (pH 8.0), 0.5% SDS, and 200 μg/ml of proteinase K and the tubes were incubated in a 55° C. water bath overnight. The next day, 500 μl of phenol:chloroform, pH 8.0, (IBI/Shelton Scientific, VWR Cat. No. IB05174) was added to the samples; tubes were mixed and then centrifuged at 15,000 rpm for 10 minutes at room temperature. The aqueous phase was extracted and placed in a new Eppendorf tube, being careful not to carry the phenol into the lower layer. To the extract was added a 2/10 volume of 10 M sodium acetate, 10 μg of glycogen (stock is 1 μg/μl) and 2.5 times the volume of 100% ethanol. The tubes were then inverted several times, incubated overnight at −20° C. and then centrifuged in a refrigerated centrifuge for 10 minutes at 15,000 rpm to collect the DNA. Supernatants were removed by decantation and tubes were inverted and blotted on paper towels. A solution of 70% ethanol was added (500 μl), the tubes were then closed and inverted to rinse the pellet. Tubes were then centrifuged in a refrigerated centrifuge for 10 minutes at 15,000 rpm, decanted of ethanol, inverted, and blotted on paper towels. Tubes were laid on their side to completely air dry and eliminate residual ethanol that could interfere with subsequent PCR reactions and DNA sequencing. The dried pellet was then resuspended in 100 μl of 10 mM Tris-HCL (pH 8.0) and incubated in a 100° C. heat block for 10 minutes to inactivate any residual proteinase K activity. Extracted DNA samples were labeled with the participant ID number and stored at −20° C. in a location recorded in the database for further applications.

The presence or the absence of HPV was assessed by real-time PCR in which the PGMY primer set was used to amplify the cervical DNA samples. The master mix for a 25 μl reaction included: 12.1 μl of water, 2.5 μl of 10× BV buffer, 2 μl of 10 mM dNTPs (Invitrogen, Cat. No. 18427-088), 1.5 μl of dimethyl sulfoxide (DMSO), 0.13 μl of Platinum Taq DNA Polymerase (Invitrogen, Cat. No. 10966-034), 0.07 μl of 100× SYBR green dye (Invitrogen, Cat. No. S-7563) and 5 μl of 2 μM Primer mix 4. BV buffer (10×) is composed of 166 mM (NH₄)₂SO₄, 670 mM Tris (pH 8.8), 67 mM MgCl₂, and 100 mM beta mercaptoethanol (β-ME). Primer mix 4 has an equal volume of each primer that has been reconstituted to a 10 μM concentration. The real-time amplification was performed using the MyIQ cycler from Bio-Rad Inc. (Cat. No. 170-9740) and a thermocycler protocol composed of 3 steps. The first step was 1 cycle of 94° C. for 2 minutes intended for initial denaturation of the DNA. The second step was the amplification that consisted of 50 cycles, each of them constituted by incubation at 94° C. for 10 seconds followed by 50 seconds at 57° C. and 50 seconds at 68° C. The third step was the melt curve beginning at 52° C. for 10 seconds and then augmenting by 0.5° C. increments up to 92° C. PCR products (10 μl) were run on a 1.5% agarose gel with 2 μl of loading dye to visualize the size of the amplified products (a 450 by amplicon of the HPV L1 gene). One lane was used for a 100 by DNA ladder to gauge the size of the PCR product (5 μl).

For determining the HPV type or types present in HPV positive samples the Inno-LiPA HPV AMP Kit (catalog #80173) and the Inno-LiPA Genotyping Extra Kit (catalog #80174) from Innogenetics were used, according to the manufacturer's recommendations. This reverse hybridization line blot assay is designed to differentiate 28 types of HPV utilizing amplification of the L1 region of the HPV genome. A primer set is used due to the variation among types in this region of the viral genome. Briefly, during PCR amplification biotinylated dNTPs are incorporated into the amplicons. Nitrocellulose strips containing immobilized complementary sequence probes for the different HPV types are incubated with a solution of denatured PCR products that results in specific binding of the biotinylated amplicons to their complementary probe on the strips. After a series of washes, strips are incubated with streptavidin-conjugated to alkaline phosphatase, which results in incorporation of this enzyme to the biotinylated amplicons hybridized on the strip. Detection of positive bands is performed by incubation of strips in a chromogenic substrate solution. Strips are washed and then dried. HPV type, or types, determination is performed by contrasting the colored bands on the nitrocellulose strip to the Inno-LiPA HPV Genotyping Extra Interpretation Chart.

Cervical exfoliated cells stored in RNAlater at −20° C. were thawed and mucus was dislodged from the cytobrush utilizing a clean 200 μl pipette tip. Cytobrushes were rotated in the cell suspension and then rubbed on the tube's wall to recover the suspension left on it as much as possible. Next, 4 ml of cold, tissue culture-grade PBS was added to each sample and tubes were inverted until complete dissolution of the crystals formed during storage. Subsequently, cells were spun down in a clinical centrifuge at speed 3 and then 2 for 3 minutes each, in that order, and the supernatant was removed by aspiration. In order to isolate RNA, the RNeasy Micro Kit (Qiagen, Cat. No. 74004) was used following the protocol suitable for fibrous tissue, according to the manufacturer's recommendations. Briefly, RLT buffer supplemented with β-ME was added to each pellet and cells were lysed by vortexing them 3 times during 10 seconds each. Cell lysates were further homogenized by spinning them through QlAshredder columns (Qiagen, Cat. No. 79656). Homogenized lysates were diluted with RNase-free water, supplemented with proteinase K (Qiagen, Cat. No. 19131) and incubated at 55° C. for 12 minutes. In the next step, ribonucleic acids (RNAs) were precipitated with ethanol and then spun through a MinElute spin column contained in the kit. RNA retained in the column was washed by centrifugation of RW1 buffer through the column. Subsequently, DNase I solution (Qiagen, Cat. No. 79254) was applied to the center of the column and after 20 minutes of incubation at room temperature they were washed again with RW1 buffer by centrifugation. RNA was further washed with RPE buffer first and with 80% ethanol in second place. Finally, the column was spun dried and RNA was eluted with RNase-free water, also by centrifugation. Samples were labeled and stored at −80° C. until used. RNA quality and quantity was evaluated in an Agilent 2100 Bioanalyzer using RNA 6000 Pico chips (Agilent, Part No. 5067-1513), according to the manufacturer's recommendations. RNA data, as well as sample storage locations, were imputed into the database.

For the purpose of RNA amplification and labeling the Amino Allyl MessageAmp II aRNA Amplification Kit (Ambion, Cat. No. AM1753) was used according to the manufacturer's recommendation. Briefly, 30 ng of total RNA was reverse transcribed for 2 hours at 42° C. using oligo(dT) primers bearing a T7 promoter, dNTPs and ArrayScript reverse transcriptase, which is engineered to produce higher yields of first-strand cDNA than wild-type reverse transcriptase. In the next step, the second DNA strand was synthesized using a dNTP mix and DNA polymerase and, at the same time, reverse transcribed RNA molecules were degraded with RNase H in a 2-hour incubation at 16° C. Synthesized double stranded DNA (dsDNA) was then purified by a series of centrifugation and washes using glass filter spin columns. Subsequently, dsDNA templates were in vitro transcribed with T7 RNA polymerase and nucleoside triphosphates (NTPs) to generate hundreds to thousands of antisense amplified RNA (aRNA) copies of each mRNA in the sample. Amplified RNA was then purified by a series of centrifugation and washes using glass filter spin columns. Given the very small quantity of starting RNA (30 ng), a second round of amplification was required. The same procedure was repeated with 2 modifications. The first change was the use of random hexamers, instead of T7 oligo(dT) primers, in the synthesis of first-strand DNA. The second modification was the change of UTP, one of the NTPs used in the previous in vitro transcription, by the modified nucleotide 5-(3-aminoallyl)-UTP (aaUTP), which contains a reactive primary amino group that can be chemically coupled to N-hydroxysuccinimidyl (NHS) ester-derivatized reactive dyes, such as cyanine 3 (Cy3) and cyanine 5 (Cy5). These changes rendered aminoallyl-modified aRNA (aaRNA) that later were labeled using Cy5 NHS ester dye (Amersham Biosciences, Cat. No. Q15104), thus generating fluorescently labeled molecules. Similarly, 1 μg of Universal Reference RNA (Stratagene, Cat. No. 740000), which consists of RNA pooled from 10 cancer cell lines, was 1-round amplified and then labeled with Cy3 NHS ester dye (Amersham Biosciences, Cat. No. Q13104). Labeled RNA samples were purified by a series of centrifugation and washes using glass filter spin columns. Subsequently, dye incorporation was evaluated and labeled samples were aliquoted and stored at −80° C. until used. Only samples with incorporation rates of 30 to 60 dye molecules per 1000 nucleotides were used, as recommended by the manufacturer.

Microarrays were printed using a non-contact Packard BioChip Arrayer (Perkin Elmer) on UltraGAPS amino silane-coated glass slides (Corning, Cat. No. 40015) using 60-mer oligonucleotides dissolved in 3× saline-sodium citrate (SSC) buffer at a 12.5 μM concentration. A total of 1204 spots were printed in 4 subarrays, each of whom had a 24×13 format. In each subarray, spots were vertically and horizontally 500 μm apart from each other and were, on average, 160 μm in diameter. The number of unique genes represented in the array was 1050 and some genes were printed more than once. Of these genes, 613 were selected from a database built by searching the literature for the genes that had been reported to be altered at the expression or genetic levels in cervical cancer. Four hundred other genes were included in the array because they were found to be differentially expressed among the stages of our in vitro model of HPV 16-mediated transformation in microarray experiments. Since differences in immunologic responses may be involved in determining HPV persistence or clearance, 37 cytokine genes were also included in the design. In addition, a GenePix Array List (GAL) files was created, which contains information about the array design including; dimensional arrangement of sub-arrays and spots as well as gene locations within the array and annotations. This file was later used during quantitation of the images to correctly link each spot florescent signal with the corresponding gene. In order to hybridize labeled samples and RNA reference to the described arrays the Pronto! Hybridization Kit was utilized (Corning, Cat. No. 40028) and the MAUI 4-bay Hybridization System (BiomicroSystem, Cat. No. 02-A002-02). Before hybridization, MAUI SC mixers (BiomicroSystem, Cat. No. 02-A008-16) were adhered to the glass microarrays forming low volume chambers for incubation of the amplified, labeled RNA samples. In addition, MAUI mixers have 2 bladders at their ends that are synchronously inflated and deflated during incubation allowing continuous mixing of the samples. Equal amounts of Cy5 labeled sample and Cy3 labeled reference RNA were mixed in Pronto! Long Oligo/cDNA Hybridization solution and incubated at 95° C. for 5 minutes. The samples were centrifuged at 13,500 g for 2 minutes and then loaded into the mixing chambers. After sealing the loading holes, they were clamped in the MAUI hybridization station that was previously equilibrated at 42° C. and hybridization was started at the lowest mixing speed. After 16-hour hybridizations, samples were washed using the buffers in the Pronto! Hybridization Kit, according to the manufacturer's recommendations, and spun dried.

Immediately after dried, hybridized arrays were scanned for both the Cy3 and Cy5 channels at 10 μm resolution using a ScanArray 5000 XL microarray scanner (Perkin Elmer Life and Analytical Sciences) and the ScanArray Express SP3 software. The scanned images were saved as TIFF files and fluorescence intensities were quantitated using the same software and the GAL files previously described. An adaptive threshold algorithm was used in the quantitation protocol to determine the edges of the spots. Results were exported into a tab-delimited text file in a GPR format for further analysis.

For data analysis, limma and limmaGUl packages were downloaded from BioConductor and used for background correction and data normalization. Both of them run under R, a freely-distributed statistical computing and graphics software. Raw intensities for backgrounds and foregrounds (spots) contained in the GPR files were uploaded into limmaGUl and background corrected using the normexp method with offset equal to 50. Subsequently, data was normalized within arrays using the print-tip locally weighted scatterplot smoothing (LOESS) algorithm. Normalization is important to correct imbalances between RNA samples that occur for a variety of technical reasons, such as differences in the setting of the PMT voltage during scanning, inequity in the total amount of labeled RNA available in each sample or differences in dye incorporation during labeling. Additionally, data was scale normalize between the arrays to correct different spreads of M-values. Normalized data (M and A values) were exported from limmaGUI in a tab-delimited text file format and normalized intensities for both Cy3 and Cy5 channel were calculated for all arrays by solving the equations for M and A, being

$$M = {log}_{2} \frac{R}{G} ) .$$

The letter R in the equations is Cy5 channel intensity (Red) and G represents Cy3 channel intensity (Green). In the next step, normalized intensities were uploaded into GeneSifter analysis software (Geospiza, Inc.) where the different sample groups were contrasted. In order to determine significance of differentially expressed genes the non-parametric Wilcoxon rank-sum test statistic was used together with a fold change cutoff value of 2 (either up or down) for the degree of intensity change between the compared groups. Also using GeneSifter software, KEGG (Kyoto Encyclopedia of Genes and Genomes) pathway analysis was performed to the list of differentially expressed genes. Affected pathways were picked on the basis of the calculated z-score that indicates whether a pathway occurs more or less frequently than just expected by chance. Z-scores greater than 2 or less than −2 were considered significant.

Differentially expressed genes for most of the group comparisons were analyzed by unsupervised agglomerative hierarchical cluster analysis. For this purpose, the software named Cluster was utilized. Limma/limmaGUI normalized data was properly formatted, imputed into Cluster and pre-processed as described in the software's manual. During cluster calculations 4 different similarity metrics (distances) were used including centered and uncentered correlation, Spearman Rank Correlation and Kendall's Tau algorithms. In addition, each of these distances was used in conjunction with each of the 3 possible linkage functions to calculate the clusters yielding 12 different ways of computing the clusters. This approach was taken to corroborate the reproducibility of the calculated clusters since different algorithms can render distinct clusters depending on the equation utilized, especially if the data does not have a strong structure. Using a nonparametric Wilcoxon rank-sum test and 0.01 and 1.5 as p-value and fold change cutoffs, respectively, 67 differentially expressed genes in HPV16 persisters as compared to HPV16 non-persisters as illustrated in Table I.

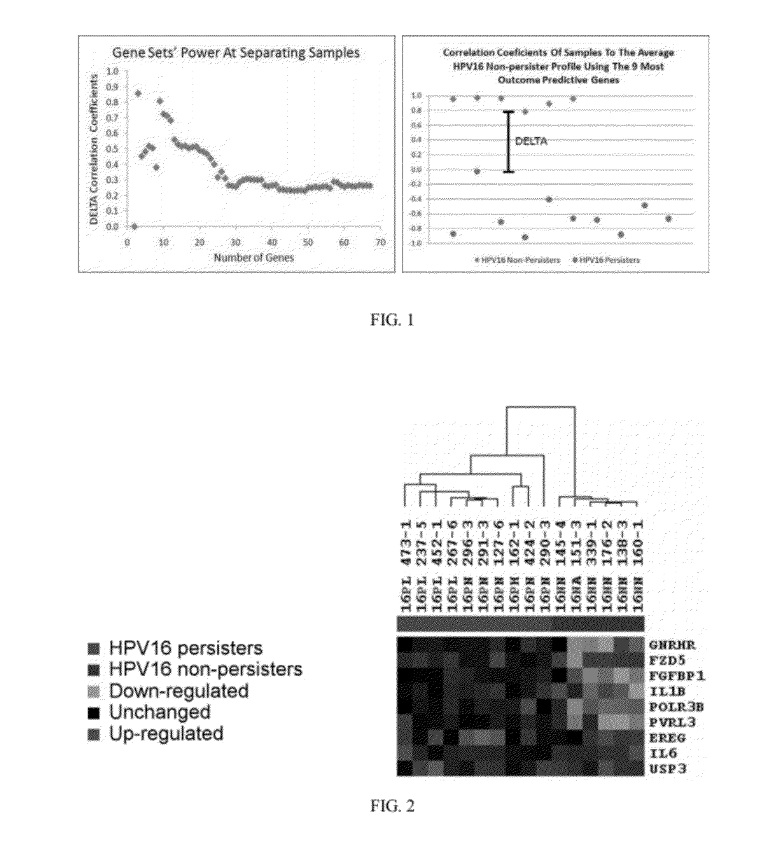

Another analysis performed on the significantly changed genes for the comparison between HPV16 persisters and non-persisters was the search of the genes with the highest outcome classification power and eliminate the ones that do not contribute to group categorization. First, outcome predictive power (OPP) for each gene was individually assessed by calculating the Pearson's correlation coefficient (CC) between the median centered M values and the outcomes, as determined by HPV testing and typing. Next, absolutes values of the coefficients were calculated and then genes were sorted in decreasing order (decreasing OPP). In addition, average HPV16 persister and non-persister profiles were separately calculated by computing the mean of the median centered M values for each gene in the two mentioned groups. Subsequently, starting with the 2 top genes, the Pearson's CC coefficient between each sample and the average HPV16 non-persister profile was calculated. While HPV16 non-persistent samples positively correlate with the average HPV16 non-persister profile, HPV16 persistent samples negatively correlate with it. Then, to assess how well the 2 groups were separated using only the 2 top genes, the difference between the smaller positive CC in the HPV16 non-persister group and the larger negative CC in the HPV16 persister group was calculated, which was called DELTA correlation coefficients. This DELTA represents the minimum separation between any 2 samples in the compared groups. The next step was to repeat the same algorithm adding one more gene at a time until inclusion of the all differentially expressed genes. Finally, the optimal number of genes for separating the samples in their outcome groups was determine by observing the maximum in the curve resulting from plotting the DELTAs vs. the numbers of most informative genes used to calculate it. The same analysis was repeated using the average HPV16 persister profile, instead of the average HPV16 non-persister profile. By outcome correlation analysis of the 67 differentially expressed genes, it was determined that the 9 most outcome predictive genes have the highest power at classifying samples (delta is maximum) into their corresponding outcome groups (illustrated in FIG. 1). Unsupervised hierarchical cluster analysis using the 9 most outcome predictive genes perfectly classified samples into their corresponding outcome groups (illustrated in FIG. 2).

Optimization of the RNA isolation protocol allowed for microarray hybridizations using samples representing 4 biologically different groups and also allowed for several comparisons. The first comparison was between HPV16 persister and HPV16 non-persister samples. Categorization of them by KEGG pathway analysis with respect to the most relevant pathways mainly indicated that genes involved in immune and inflammatory responses were down-regulated in HPV persistent samples. Among them are:
Cytokines CCL2 and CCL17 that are involved in chemotactic activity for monocytes and basophils and for T lymphocytes, respectively.
IL1A that is released in response to cell injury induces apoptosis and is involved in various immune responses, inflammatory processes, and hematopoiesis.
IL6 that has different functions in inflammation and is involved in the maturation of B cells.
TNF that is a multifunctional pro-inflammatory cytokine plays a role in wide spectrum of biological processes including cell proliferation, differentiation, apoptosis, lipid metabolism, and coagulation.
TLR8 that is involved in pathogen recognition and activation of innate immunity and mediates the production of cytokines necessary for the development of effective immunity.

Two additional genes involved in immune and inflammatory processes were found up-regulated. They are IL1B that has biological functions similar to IL1A and cytokine CXCL1 that is involved in neutrophil chemotaxis and has been reported to have mitogenic properties in human melanoma cells. Gene functions were obtained from the Online Mendelian Inheritance in Man (OMIM) database.

The next step was to evaluate how well the significantly changed genes could classify HPV16 persistent and HPV16 non-persistent patients. Further analysis and selection of the 9 most informative genes resulted in a perfect separation of the samples in their corresponding groups as evidenced by the correlation coefficients of them to the average prognostic profiles (FIG. 1) and by cluster analysis (FIG. 2). The discovery of this molecular signature has an immense potential clinical importance since it allows prediction of whether or not HR-HPV infections will be persistent or will be cleared. Consequently, women will be able to be triaged more accurately for further follow up and treatment reducing the drawbacks of the current cytological and histological diagnostic methods.

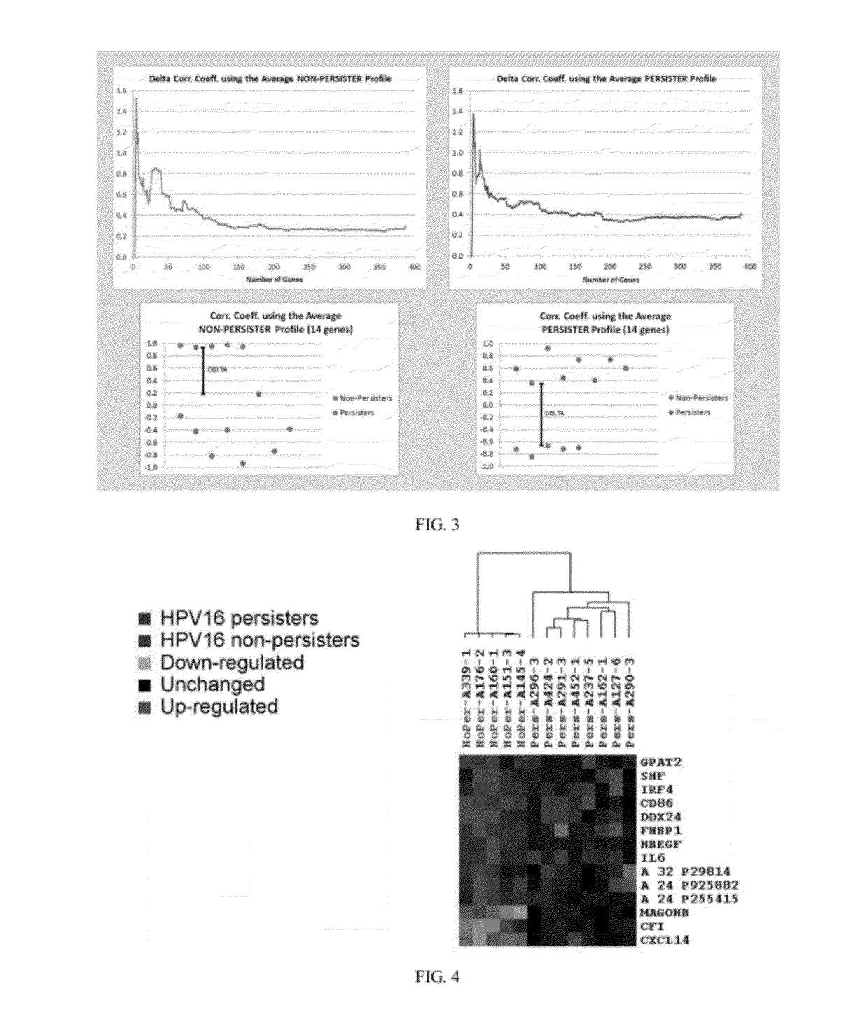

A more extensive analysis, using microarrays containing approximately 40,000 potential biomarkers identified additional biomarker genes that may be even more predictive of persistent HR-HPV infection. The same samples utilized in the previous example were utilized with a human commercial array from Agilent Technologies [part number G4112F (4X44K)] which contains probes for over 41,000 unique genes and transcripts. A larger set of differentially expressed genes shown in Table II was generated. A profile of genes (14 different genes) that allowed the separation of the samples that were from HPV16 non-persisters and HPV16-persisters. Analysis and selection of the 14 most informative genes resulted in a separation of the samples in their corresponding groups as evidenced by the correlation coefficients of them to the average prognostic profiles (FIG. 3) and by cluster analysis (FIG. 4). These 14 genes only contain one gene (IL6) that overlaps the 9 genes described in the previous example. This is not unexpected since the array had so many more genes.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A method for the diagnosis of persistent HR-HPV in an individual comprising the following steps:
(a) obtaining a biological sample from an individual;
(b) determining an expression level of at least one biomarker in the biological sample,
wherein the at least one biomarker is chosen from GPAT2, SHF, IRF4, CD86, DDX24, FNBP1, HBEGF, IL6, A 32 P29814, A 24 P925882, A 24 P255415, MAGOHB, CFI, and CXCL14;
(c) comparing the expression level of the at least one biomarker to an expression level of one or more corresponding biomarkers in a comparative sample, wherein the comparative sample contains the at least one biomarker in a level indicative of persistent HR-HPV; and
(d) predicting persistent HR-HPV infection in the individual based upon the comparison of the expression level of the at least one biomarker between the comparative sample and the biological sample.

2. The method of claim 1, wherein the biomarker is GPAT2.

3. The method of claim 1, wherein the biomarker is SHF.

4. The method of claim 1, wherein the biomarker is IRF4.

5. The method of claim 1, wherein the biomarker is CD86.

6. The method of claim 1, wherein the biomarker is DDX24.

7. The method of claim 1, wherein the biomarker is FNBP1.

8. The method of claim 1, wherein the biomarker is HBEGF.

9. The method of claim 1, wherein the biomarker is IL6.

10. The method of claim 1, wherein the biomarker is A 32 P29814.

11. The method of claim 1, wherein the biomarker is A 24 P925882.

12. The method of claim 1, wherein the biomarker is A 24 P255415.

13. The method of claim 1, wherein the biomarker is MAGOHB.

14. The method of claim 1, wherein the biomarker is CFI.

15. The method of claim 1, wherein the biomarker is CXCL14.

16. The method of claim 1, wherein the biological sample comprises cells from a PAP test.

17. A method for the diagnosis of persistent HR-HPV in an individual comprising the following steps:
(a) obtaining a biological sample from an individual;
(b) determining an expression level of at least one biomarker in the biological sample, wherein the at least one biomarker is chosen from GNRHR, FZDS, FGFBP1, IL1B, POLR3B, PVRL3, EREG, IL6, and USP3;
(c) comparing the expression level of the at least one biomarker to an expression level of one or more corresponding biomarkers in a comparative sample, wherein the comparative sample contains the at least one biomarker in a level indicative of persistent HR-HPV; and
(d) predicting persistent HR-HPV infection in the individual based upon the comparison of the expression level of the at least one biomarker between the comparative sample and the biological sample.

18. The method of claim 17, wherein the biological sample comprises cells from a PAP test.
